(19) United States
(12) Patent Application Publication
(10) Pub. No.: US 2023/0008427 A1
(43) Pub. Date: Jan. 12, 2023

(54) COSMETIC COMPOSITION COMPRISING AT LEAST ONE POLAR OIL, AN ALIPHATIC MONOALCOHOL, A MIXTURE OF POLYOLS AND AT LEAST ONE HYDROPHILIC ACTIVE AGENT

(71) Applicant: L’OREAL, Paris (FR)

(72) Inventors: Arno WAHLER, Chevilly La Rue (FR); Muriel ISOIR-INGREZ, Chevilly La Rue (FR); Sophie GUILBAUD, Gigors Et Lozeron (FR); Valérie PAGE, Gigors Et Lozeron (FR); Jean-Yves FOURON, Chevilly La Rue (FR); Pamella WANG, Chevilly La Rue (FR)

(21) Appl. No.: 17/785,175

(22) PCT Filed: Dec. 18, 2020
(86) PCT No.: PCT/EP2020/087027

§ 371 Date: Jun. 14, 2022

(30) Foreign Application Priority Data
Dec. 20, 2019 (FR) FR1915200

(51) Int. Cl. A61K 8/92; A61K 8/34; A61K 8/36; A61Q 5/00

(52) U.S. Cl. CPC A61K 8/92; A61K 8/34; A61K 8/361; A61Q 5/002

(57) ABSTRACT

The present invention relates to a cosmetic composition comprising:
from 30% to 89% by weight of at least one polar oil with respect to the total weight of the composition;
from 6% to 45% by weight of at least one C₂-C₆ aliphatic monoalcohol with respect to the total weight of the composition;
from 4% to 30% by weight of hexylene glycol with respect to the total weight of the composition;
from 8% to 40% by weight of dipropylene glycol with respect to the total weight of the composition; and at least one hydrophilic active agent,
said composition containing less than 15% by weight of water with respect to the total weight of the composition.

The present invention relates to a cosmetic composition, comprising less than 15% by weight of water, and comprising:
from 30% to 89% by weight of at least one polar oil with respect to the total weight of the composition;
from 6% to 45% by weight of at least one C₂-C₆ aliphatic monoalcohol with respect to the total weight of the composition;
from 4% to 30% by weight of hexylene glycol with respect to the total weight of the composition;
from 8% to 40% by weight of dipropylene glycol with respect to the total weight of the composition; and at least one hydrophilic active agent. The present invention also relates to the cosmetic use of such a composition, as well as a non-therapeutic cosmetic keratin material care method using such a composition.

In the cosmetic field of care, cosmetic compositions making it possible to obtain both good sensory properties as well as a care effect are increasingly sought by users. Care effect denotes for example an effect countering the drying or aging of keratin materials and particularly the skin, this care effect being provided by active agents. As such, hydrophilic agents, for example of C-glycoside derivative type, prove to be particularly of interest; in particular, these compounds are particularly effective for treating skin aging problems.

Furthermore, oily dosage forms are of interest for enhancing the delivery of an active agent for application on keratin materials, preferably the skin, and thus the efficacy thereof, in particular the efficacy thereof for treating skin aging problems. Oily dosage forms are also increasingly sought in the cosmetic field and more specifically in the field of skin care, particularly as they provide nourishment after application.

However, obtaining this dual effect (good sensory properties and care effect) requires, on one hand, being able to introduce one (or more) hydrophilic active agent(s) in sufficient quantity in the composition, and, on the other, overcoming the drawbacks associated with oily dosage forms. Yet, the hydrophilic nature of said active agent makes it difficult to incorporate this type of compound in oily dosage forms, which generally comprise less than 15% by weight of water.

Consequently, there remains a need for cosmetic compositions rich in oil(s), and comprising an effective quantity of hydrophilic active agent(s), which help improve the delivery of these active agents and increase the bioavailability thereof during application on keratin materials, which are stable, and which have good sensory properties, particularly which increase emollience after application, while retaining the advantageous properties of oils, in particular nourishment.

The term “bioavailability” denotes, for the purposes of this application, the molecular penetration of the active agent concerned in the living layers of the skin and in particular the epidermis. It will be sought to ensure that the penetrated concentration is as high as possible.

The Applicant surprisingly observed that an oily cosmetic composition comprising at least one polar oil, a C₂-C₆ aliphatic monoalcohol and an association of specific polyols, in defined contents, and at least one hydrophilic active agent, makes it possible to enhance the emollience of the composition and obtain good penetration and bioavailability of the hydrophilic active agent after application, while being stable.

Thus, the invention also relates to a cosmetic composition comprising:
from 30% to 89% by weight of at least one polar oil with respect to the total weight of the composition;
from 6% to 45% by weight of at least one C₂-C₆ aliphatic monoalcohol with respect to the total weight of the composition;
from 4% to 30% by weight of hexylene glycol with respect to the total weight of the composition;
from 8% to 40% by weight of dipropylene glycol with respect to the total weight of the composition; and
at least one hydrophilic active agent,
said composition comprising less than 15% by weight of water with respect to the total weight of the composition.

The composition according to the invention is stable.

A composition is referred to as stable when the macroscopic appearance (clearness and homogeneity) does not change after at least 24 hours. In particular, the composition according to the invention has a clear and homogeneous appearance.

For the purposes of this invention, the term “clear” denotes a composition having a transparent appearance.

For the purposes of this invention, the term “homogeneous” denotes a composition consisting of a single phase, in other words mono-phase.

The composition according to the invention is preferably intended for a topical application, particularly on keratin materials, in particular the skin. It particularly comprises a physiologically acceptable medium, i.e., a medium compatible with all keratin materials, in particular the skin.

For the purposes of this invention, the term keratin materials denotes the skin and the appendages thereof.

The term “skin” denotes facial skin and/or body skin, and the scalp.

The term “appendages” denotes eyelashes, eyebrows, nails and hair, and particularly eyelashes and hair.

The present invention also relates to a method of cosmetic treatment of keratin materials, comprising application of a composition according to the invention on said keratin materials. Moreover, the invention also relates to the use of said composition in the cosmetic field, in particular for body or facial skin care.

Preferably, the composition according to the invention comprises less than 5% by weight of surfactant, preferably less than 3% by weight of surfactant, more preferentially less than 2% by weight of surfactant, preferably less than 1% by weight of surfactant with respect to the total weight of the composition. Preferably, the composition is free from surfactant.

For the purposes of this invention, the term “surfactant” denotes an amphiphilic molecule, i.e., it has two parts of different polarity, in general one is lipophilic (soluble dispersible in an oily phase), the other is hydrophilic (soluble or dispersible in water). Surfactants are characterized by the value of the HLB (Hydrophilic Lipophilic Balance) thereof, the HLB being the ratio between the hydrophilic part and the lipophilic part in the molecule. The term HLB is well known to those skilled in the art and is described for example in “The HLB system. A time-saving guide to Emulsifier Selection” (published by ICI Americas Inc; 1984). For emulsifying surfactants, the HLB generally ranges from 3 to 8 for the preparation of W/O emulsions and from 8 to 18 for the preparation of O/W emulsions. The HLB of the surfactant(s) used according to the invention can be determined using the GRIFFIN method or the DAVIES method.

Preferably, the composition according to the invention does not comprise emulsifying surfactants such as polyoxyethylenated castor oil, fatty acid and sorbitol and/or sorbitan esters optionally oxyethylenated such as polysorbates, phospholipids, polyethylene glycol and fatty acid esters such as PEG-15 stearate, as well as lecithin.

The composition according to the invention comprises less than 15% by weight of water with respect to the total weight of the composition, preferably less than 8% by weight of water with respect to the total weight of the composition, preferably less than 5% by weight of water with respect to the total weight of the composition, preferably less than 4% by weight of water with respect to the total weight of the composition.

Preferably, the composition according to the invention is free from water.

The composition according to the invention differs in particular from emulsions in that it is not presented in the form of a dispersion of two mutually non-miscible liquids at ambient temperature (20-25° C.). The composition according to the invention is indeed single-phase. It has no dispersed phase (also known as discontinuous phase) in droplet form in a dispersing phase (also known as continuous phase). The macroscopic analysis (with the naked eye) of the composition according to the invention shows a transparent and single-phase appearance.

### POLAR OIL

The composition according to the invention comprises from 30% to 89% by weight of at least one polar oil with respect to the total weight of the composition. Preferably, the composition according to the invention comprises from 35% to 80% by weight of at least one polar oil, from 40% to 75% by weight of at least one polar oil with respect to the total weight of the composition.

The term “oil” denotes any fatty substance in liquid form at ambient temperature (25° C.) and at atmospheric pressure. Among the oils which can be used in the present invention, mention can be made of: volatiles or non-volatile oils, these oils can be hydrocarbon oils particularly of animal or plant origin, synthetic oils, silicone oils, fluorinated oils, or mixtures thereof.

For the purposes of this invention, the term “silicone oil” denotes an oil comprising at least one silicon atom, and in particular at least one Si-O group.

The term “hydrocarbon oil” denotes an oil containing essentially hydrogen and carbon atoms and optionally oxygen, nitrogen, sulfur and/or phosphorus atoms.

The term “polar oil”, for the purposes of this invention, denotes an oil wherein the solubility parameter δₐ at 25° C. is different to 0 (J/cm³)^{½}. In particular, the term “polar oil” denotes an oil wherein the chemical structure is essentially formed from, or consists of, carbon and hydrogen atoms, and comprising at least one highly electronegative heteroatom such as an oxygen, nitrogen, silicon or phosphorus atom.

The definition of HANSEN three-dimensional solubility parameters are described in the article by C. M. HANSEN: “The three dimensional solubility parameters” J. Paint Technol, 39, 105 (1967).

According to the Hansen space:
δₒ characterizes the LONDON dispersion forces derived from the formation of dipoles induced during molecular shocks;
δₚ characterizes the DEBYE interaction forces between permanent dipoles and the KEESOM interaction forces between induced dipoles and permanent dipoles;
δₕ characterizes the specific interaction forces (such as hydrogen, acid/base, donor/acceptor bonds, etc.);
δₐ is determined by the equation:

$$δ \text{a =} \left(δ {\text{p}}^{2} + δ {\text{h}}^{2}\right) \frac{1}{2}$$

The parameters δₚ, δₕ, δ_{D}, and δₐ are expressed in (MPa)^{½}, a unit equivalent to (J/cm³)^{½}. The Hansen solubility parameters are calculated using HSPiP software version V4.1 based on a chemical structure; the method selected in the software is Y-MB or Yamamoto-Molecular Break.

This software is available to download from the official Hansen parameter and HSPiP software website at the address www.hansen-solubility.com.

Preferably, the polar oils used according to the present invention have a δₐ between 4 and 9.1, preferably a δₐ between 6 and 9.1, more preferably between 7.3 and 9.1.

### Non-Volatile Polar Oils

For the purposes of this invention, the term “non-volatile oil” denotes an oil having a vapor pressure less than 0.13 Pa (0.01 mm Hg), at ambient temperature and atmospheric pressure. The non-volatile oils can particularly be selected from non-volatile hydrocarbon oils, where applicable fluorinated, and/or silicone oils.

By way of non-volatile hydrocarbon oil suitable for the implementation of the invention, mention can particularly be made of:
hydrocarbon oils of animal origin,
hydrocarbon oils of plant origin, such as phytostearyl esters, such as phytostearyl oleate, phytostearyl isostearate and lauroyl/octyldodecyl/phytostearyl glutamate, for example sold under the trade name ELDEW PS203Ⓡ by AJINOMOTO, triglycerides constituted of esters of fatty acids and glycerol for which the fatty acids can have chain lengths ranging from C₄ to C₂₄, with the latter able to be linear or branched, saturated or unsaturated; these oils are particularly heptanoic or octanoic oils, wheat germ, sunflower, grape seed, sesame, corn, apricot, castor, camelina, shea, avocado, olive, soybean, sweet almond, palm, rapeseed, cotton, hazelnut, macadamia, jojoba, alfalfa, poppy seed, pumpkin, squash, blackcurrant, evening primrose, millet, barley, quinoa, rye, safflower, candlenut, passiflora, musk rose oils, or caprylic/capric acid triglycerides such as those sold by Stearineries Dubois or those sold under the trade names Miglyol 810, 812 and 818® by Dynamit Nobel, refined plant perhydrosqualene sold under the trade name Fitoderm by Cognis.

By way of example of castor oil according to the invention, mention can more specifically be made of castor oil comprising at least 50% by weight of linear or branched, saturated or unsaturated, C₁₄-C₂₂ fatty acids, preferably at least 70% by weight of linear or branched, saturated or unsaturated, C₁₆-C₂₀ fatty acids, more preferably at least 50% by weight of linear or branched, saturated or unsaturated, C₁₈ fatty acids, of which less than 5% by weight of saturated C₁₈ fatty acids, preferably less than 2.5% by weight of saturated C₁₈ fatty acids such as stearic acid, 50% to 98% by weight of monounsaturated C₁₈ fatty acids, more preferentially 80% to 98% by weight of monounsaturated C₁₈ fatty acids, such as ricinoleic and oleic acids, less than 10% by weight of polyunsaturated C₁₈ fatty acids, preferably from 2% to 8% of polyunsaturated C₁₈ fatty acids such as linoleic and linolenic acids, with respect to the total weight of the fatty acids present in said castor oil; such a castor oil is sold by VERTELLUS under the name CRYSTAL O.

By way of example of camelina oil according to the invention, mention can more specifically be made of camelina oil comprising at least 50% by weight of linear or branched, saturated or unsaturated, C₁₄-C₂₂ fatty acids, preferably at least 70% by weight of linear or branched, saturated or unsaturated, C₁₆-C₂₀ fatty acids, more preferably at least 50% by weight of linear or branched, saturated or unsaturated, C₁₈ fatty acids, of which less than 5% by weight of saturated C₁₈ fatty acids, 12% to 26% by weight of monounsaturated C₁₈ fatty acids, such as oleic acid, from 35% to 64% by weight of polyunsaturated C₁₈ fatty acids such as linoleic and linolenic acids, with respect to the total weight of the fatty acids present in said castor oil; such a camelina oil is sold by GREENTECH under the name CAMELINA REFINED OIL C05002.

By way of example of caprylic/capric acid triglycerides, mention can be made of those comprising from 45% to 80% by weight of C₈ acids and from 20% to 45% by weight of C₁₀ fatty acids, and particularly those sold by PT MUSIM MAS.
synthetic esters such as oils of formula R₁COOR₂, wherein R₁ represents a linear or branched fatty acid residue containing from 1 to 40 carbon atoms, and R₂ represents a hydrocarbon-based chain, particularly a branched chain, containing from 1 to 40 carbon atoms provided that the sum of R₁ and R₂ is greater than or equal to 10. The esters can particularly be selected from esters, particularly fatty acid esters, such as for example:
cetostearyl octanoate, isopropyl alcohol and C8-C18, preferably C12-C16, fatty acid esters, such as isopropyl myristate, isopropyl palmitate, ethyl palmitate, 2-ethylhexyl palmitate, isopropyl stearate or isostearate, isostearyl isostearate, octyl stearate, hydroxylated esters, for instance isostearyl lactate, octyl hydroxystearate, diisopropyl adipate, heptanoates, and particularly isostearyl heptanoate, alcohol or polyalcohol octanoates, decanoates or ricinoleates, such as propylene glycol dioctanoate, cetyl octanoate, tridecyl octanoate, 2-ethylhexyl 4-diheptanoate and palmitate, alkyl benzoate, polyethylene glycol diheptanoate, propylene glycol 2-diethylhexanoate, and mixtures thereof, C₁₂-C₁₅ alkyl benzoates, hexyl laurate, neopentanoic acid esters, for instance isodecyl neopentanoate, isotridecyl neopentanoate, isostearyl neopentanoate, or octyldodecyl neopentanoate, isononanoic acid esters, such as isononyl isononanoate, isotridecyl isononanoate, octyl isononanoate, hydroxylated esters such as isostearyl lactate, diisostearyl malate;
polyol esters, and pentaerythritol esters, such as dipentaerythrityl tetrahydroxystearate/tetraisostearate,
esters of diol dimers and diacid dimers, such as Lusplan DD-DA5^{®} and Lusplan DD-DA7^{®}, sold by the NIPPON FINE CHEMICAL COMPANY and described in the application FR 03 02809,
fatty alcohols that are liquid at ambient temperature, with a branched and/or unsaturated carbon chain having from 12 to 26 carbon atoms, preferably 16 to 22 carbon atoms, more preferably from 18 to 20 carbon atoms, such as 2-octyldodecanol, isostearyl alcohol, oleic alcohol, 2-hexyldecanol, 2-butyloctanol, and 2-undecylpentadecanol,
higher fatty acids, such as oleic acid, linoleic acid, linolenic acid, and mixtures thereof,
dialkyl carbonates, the 2 alkyl chains possibly being identical or different, such as the dicaprylyl carbonate sold under the trade name Cetiol CC®, by Cognis,
diesters of C2-C16, preferably C8-C12, dicarboxylic acid and C1-C4 monoalcohol, preferably of C3-C4 branched monoalcohol; preferably the diester of sebacic acid and isopropyl alcohol such as the diisopropyl sebacate sold under the name DUB DIS by STÉARINERIES DUBOIS,
the non-volatile silicone oils, for example non-volatile polydimethylsiloxanes (PDMS), polydimethylsiloxanes comprising alkyl or alkoxy groups which are pendant and/or at the end of the silicone chain, groups each having from 2 to 24 carbon atoms, phenylated silicones such as phenyl trimethicones, phenyl dimethicones, phenyltrimethylsiloxydiphenylsiloxanes, diphenyl dimethicones, diphenylmethyldiphenyltrisiloxanes, and (2-phenylethyl)trimethylsiloxysilicates, dimethicones or phenyltrimethicone of viscosity less than or equal to 100 Cst, and
mixtures thereof.

### Volatile Polar Oils

For the purposes of this invention, the term “volatile oil” denotes an oil (or nonaqueous medium) liable to evaporate on contact with the skin in less than one hour, at ambient temperature and atmospheric pressure. The volatile oil is a volatile cosmetic oil, liquid at ambient temperature, particularly having a vapor pressure different to zero, at ambient temperature and atmospheric pressure, particularly having a vapor pressure ranging from 0.13 Pa to 40,000 Pa (10⁻³ at 300 mm Hg), in particular ranging from 1.3 Pa to 13,000 Pa (0.01 to 100 mm Hg), and preferentially ranging from 1.3 Pa to 1300 Pa (0.01 at 10 mm Hg).

It is possible to use, as volatile oils, volatile silicones, such as, for example, volatile linear or cyclic silicone oils, in particular those having a viscosity below or equal to 8 centistokes (8.10⁻⁶ m²/s), and having particularly from 2 to 10 silicon atoms, and in particular from 2 to 7 silicon atoms, these silicones optionally comprising alkyl or alkoxyl groups having from 1 to 10 carbon atoms. By way of volatile silicone oil that can be used in the invention, mention can be made particularly of dimethicones with a viscosity of 5 and 6 cSt, octamethyl cyclotetrasiloxane, decamethyl cyclopentasiloxane, dodecamethyl cyclohexasiloxane, heptamethyl hexyltrisiloxane, heptamethyloctyl trisiloxane, hexamethyl disiloxane, octamethyl trisiloxane, decamethyl tetrasiloxane, dodecamethyl pentasiloxane, and mixtures thereof. Volatile fluorinated oils such as nonafluoromethoxybutane or perfluoromethylcyclopentane and mixtures thereof can also be used. A mixture of the oils cited above can also be used.

Preferably, the polar oil is non-volatile.

According to a preferred embodiment, the polar oil(s) is/are selected from hydrocarbon oils of plant origin, synthetic esters of formula R₁COOR₂ wherein R₁ represents a linear or branched fatty acid residue containing from 1 to 40 carbon atoms, and R₂ represents a hydrocarbon-based chain, particularly a branched chain, containing from 1 to 40 carbon atoms provided that the sum of R₁ and R₂ is greater than or equal to 10, fatty alcohols that are liquid at ambient temperature, with a branched and/or unsaturated carbon chain having from 12 to 26 carbon atoms, dialkyl carbonates, diesters of C₂-C₁₆ dicarboxylic acid and C₁-C₄ monoalcohol; and mixtures thereof.

Preferably, the polar oil(s) is/are selected from triglycerides constituted of esters of glycerol and C₄ to C₂₄ fatty acids that are linear or branched, saturated or unsaturated; esters of isopropyl alcohol and C₈-C₁₈, preferably C₁₂-C₁₆, fatty acids; fatty alcohols that are liquid at ambient temperature, with a branched and/or unsaturated carbon chain having from 16 to 22 carbon atoms, preferably from 18 to 20 carbon atoms; dialkyl carbonates, both alkyl chains being identical, preferably dicaprylylcarbonate; diesters of C₈-C₁₂ dicarboxylic acid and branched C₈-C₄ monoalcohol, preferably diisopropyl sebacate; and mixtures thereof.

The polar oil(s) within the scope of the present invention can be selected preferably from triglycerides constituted of esters of glycerol and C₄ to C₂₄ fatty acids that are linear or branched, saturated or unsaturated, esters of isopropyl alcohol and C₈-C₁₈ fatty acids, fatty alcohols that are liquid at ambient temperature, with a branched and/or unsaturated carbon chain having from 16 to 22 carbon atoms, and mixtures thereof.

The polar oil(s) within the scope of the present invention can be selected preferably from triglycerides of glycerol and C₆-C₁₂ fatty acids, triglycerides of glycerol and C₁₄-C₂₂ fatty acids, esters of isopropyl alcohol and C₈-C₁₈ fatty acids, fatty alcohols that are liquid at ambient temperature, with a branched and/or unsaturated carbon chain having from 18 to 20 carbon atoms, and mixtures thereof.

Preferentially, the triglycerides of glycerol and C₁₄-C₂₂ fatty acids comprise from 50% to 100% by weight of linear, branched, saturated or unsaturated C₁₈ fatty acids of which 0% to 5% by weight of saturated C₁₈ fatty acids such as stearic acid, from 50% to 98% by weight of monounsaturated fatty acids such as ricinoleic and/or oleic acids, and/or from 2% to 70% by weight of polyunsaturated C₁₈ fatty acids such as linoleic and/or linolenic acids, with respect to the total weight of fatty acids comprised in said triglycerides.

Preferably, the triglycerides of glycerol and C₆-C₁₂ fatty acids comprise from 45% to 80% by weight of C₈ fatty acids and from 20% to 45% by weight of C₁₀ fatty acids, with respect to the total weight of fatty acids comprised in said triglycerides.

Preferably, the composition according to the invention comprises at least one polar oil selected from fatty alcohols that are liquid at ambient temperature, with a branched and/or unsaturated carbon chain having from 18 to 20 carbon atoms, triglycerides of glycerol and C₁₄-C₂₂ fatty acids and mixtures thereof. Preferably, the composition according to the invention comprises at least one polar oil selected from octyldodecanol, oleic alcohol, castor oil, camelina oil, and mixtures thereof.

Preferably, the composition according to the invention comprises at least two polar oils as defined above, different from one another, in particular, at least a first polar oil selected from fatty alcohols that are liquid at ambient temperature, with a branched and/or unsaturated carbon chain having from 18 to 20 carbon atoms, and at least a second polar oil selected from triglycerides of glycerol and C₁₄-C₂₂ fatty acids.

Preferably, the composition according to the invention comprises at least two polar oil of which at least a first polar oil selected from octyldodecanol, oleic alcohol and mixtures thereof, and at least a second oil selected from castor oil, camelina oil, and mixtures thereof.

Preferably, the composition according to the invention comprises a mixture of octyldodecanol, oleic acid, castor oil and camelina oil.

Preferably, the first polar oil selected from fatty alcohols that are liquid at ambient temperature, with a branched and/or unsaturated carbon chain having from 18 to 20 carbon atoms, is present in the composition at a concentration between 25% and 65% by weight, preferably between 35% and 45% by weight with respect to the total weight of the composition.

Preferably, the second polar oil selected from triglycerides and glycerol and C₁₄-C₂₂ fatty acids is present in the composition at a concentration between 4% and 30% by weight, preferably between 5% and 30% by weight with respect to the total weight of the composition.

### ADDITIONAL FATTY ACIDS

The composition according to the invention can comprise additional fatty acids different from the polar oils cited above, and particularly one or more non-polar oils and/or one or more solid and/or pasty fats.

The term “non-polar oil”, for the purposes of this invention, denotes an oil wherein the solubility parameter δₐ at 25° C. as defined above is equal to 0 (J/cm³)^{½}.

Among the non-polar oils, mention can be made for example of:

### Non-volatile Non-Polar Oils

Hydrocarbon oils of mineral or synthetic origin, such as for example: linear or branched hydrocarbons of mineral or synthetic origin, such as petroleum jelly, polydecenes, hydrogenated polyisobutene such as Parleam, squalane, and mixtures thereof, and in particular hydrogenated polyisobutene.

### Volatile Non-Polar Oils

Volatile hydrocarbon oils can be selected from hydrocarbon oils having from 8 to 16 carbon atoms, and particularly branched C₈-C₁₆ alkanes (also known as isoparaffins) such as isododecane (also known as 2,2,4,4,6-pentamethylheptane), isodecane, isohexadecane, and for example the oils sold under the trade names Isopars® or Permethyls®; linear alkanes, for example n-dodecane (C12) and n-tetradecane (C14) sold by Sasol respectively under the references PARAFOL 12-97 and PARAFOL 14-97, as well as the mixtures thereof, the undecane-tridecane mixture (Cetiol UT®), the mixtures of n-undecane (C11) and n-tridecane (C13) obtained in examples 1 and 2 of application WO2008/155059 of Cognis and mixtures thereof.

Preferably, the non-polar oil is selected from non-volatile non-polar oils, more preferably the non-polar oil is selected from hydrocarbon oils of mineral or synthetic origin, in particular linear or branched hydrocarbons, of mineral or synthetic origin. In a preferred embodiment, the non-polar oil is squalane.

Preferably, the composition according to the invention comprises less than 10% by weight of non-polar oil with respect to the total weight of the composition, preferentially less than 6% by weight of non-polar oil, more preferably less than 5% by weight of non-polar oil with respect to the total weight of the composition, or the composition is free from non-polar oil.

The other fats which can be present in the composition according to the invention are particularly solid and/or pasty fats.

The term “solid fat” denotes any fatty substance in solid form at ambient temperature (25° C.) and at atmospheric pressure.

The term “pasty fat” denotes a lipophilic fat compound having a reversible solid/liquid change of state, having in the solid state, an anisotropic crystalline organization, and comprising at a temperature of 23° C. a liquid fraction and a solid fraction. In other words, the initial melting point of the pasty fat can be less than 23° C. The liquid fraction of the pasty fat measured at 23° C. can represent 9 to 97% by weight of the pasty fat. This liquid fraction at 23° C. preferably represents between 15 and 85%, more preferably between 40 and 85% by weight.

The melting point is equivalent to the temperature of the most endothermic peak observed in thermal analysis (DSC) as described in the standard ISO 11357-3; 1999. The melting point of a pasty fat can be measured using a differential scanning calorimeter (DSC), for example the calorimeter sold under the name “MDSC 2920” by TA Instruments.

According to a specific embodiment of the invention, the composition according to the invention comprises less than 3% by weight of solid and/or pasty fats, preferentially less than 2% by weight of solid and/or pasty fats, more preferably less than 1% by weight of solid and/or pasty fats with respect to the total weight of the composition. Preferably, the composition is free from solid and/or pasty fats.

### C₂-C₆ MONOALCOHOL

The composition according to the invention comprises from 6% to 45% by weight of a C₂-C₆ aliphatic monoalcohol, preferably from 6% to 40% by weight, preferably from 6.5% to 30% by weight, preferably from 6% to 15% by weight of at least one C₂-C₆ aliphatic monoalcohol.

Preferentially, said aliphatic monoalcohol comprises from 2 to 4 carbon atoms.

The term “aliphatic monoalcohol” denotes any saturated, linear or branched alkane compound having a single hydroxyl (OH) function. The aliphatic monoalcohol(s) present in the compositions according to the invention can be selected from ethanol, propanol, butanol, isopropanol, isobutanol or mixtures thereof.

More specifically, ethanol will be selected.

### POLYOL MIXTURE

The composition according to the invention comprises a mixture of hexylene glycol and dipropylene glycol.

It comprises from 4% to 30% by weight of hexylene glycol, and from 8% to 40% by weight of dipropylene glycol with respect to the total weight of the composition.

Hexylene glycol and dipropylene glycol are polyols, in this case diols. The term “polyol” denotes any organic molecule that contains at least two free hydroxyl groups.

Preferably, the composition according to the invention comprises from 4% to 10% by weight of hexylene glycol, preferentially from 4.3% to 10% by weight of hexylene glycol. Preferably, it comprises from 8% to 25% by weight of hexylene glycol, preferably from 8.4% to 25% by weight of dipropylene glycol with respect to the total weight of the composition.

Preferably, the composition according to the invention has a weight ratio between the oily phase and the hydrophilic phase less than or equal to 3, preferably less than or equal to 2.7, preferably less than or equal to 2.5. Preferably, said weight ratio between the oily phase and the hydrophilic phase is greater than or equal to 0.8, preferably greater than or equal to 0.85, preferably greater than or equal to 0.9.

The oily phase according to the invention comprises the polar oil(s) according to the invention, and optionally the other lipophilic compounds. The hydrophilic phase according to the invention comprises the C₂-C₆ aliphatic monoalcohol, hexylene glycol, dipropylene glycol, the hydrophilic active agent, and optionally water.

Preferably, the weight ratio between the oily phase and the hydrophilic phase is between 0.8 and 3, preferably between 0.9 and 2.5.

### HYDROPHILIC ACTIVE AGENT

The composition comprises at least one hydrophilic active agent.

The term “hydrophilic active agent” denotes a water-soluble or water-dispersible active agent capable of forming hydrogen bonds.

By way of hydrophilic active agents, mention can be made for example of moisturizing agents; depigmenting agents, desquamating agents, anti-aging agents, matting agents; healing agents; antibacterial agents; and mixtures thereof.

Preferably, the composition according to the invention comprises from 0.01% to 20% by weight of at least one hydrophilic active agent, preferentially from 0.1% to 15% by weight, and preferentially from 1% to 1 2% by weight of at least one hydrophilic active agent with respect to the total weight of the composition.

In particular, the composition according to the invention can comprise at least one hydrophilic active agent selected from the C-glycoside derivatives of the following general formula (I):

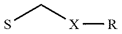

wherein:
R designates a non-substituted C₁-C₄, particularly C₁-C₂, linear alkyl radial, in particular methyl;
S represents a monosaccharide selected from D-glucose, D-xylose, N-acetyl-D-glucosamine or L-fucose, and in particular D-xylose;
X represents a group selected from —CO—, —CH(OH)—, —CH(NH₂)—, and preferentially a —CH(OH)— group;
as well the cosmetically acceptable salts thereof, solvates thereof such as hydrates and the optical isomers thereof.

By way of illustration and not limitation of the C-glycosides of formula (I) more specifically suitable for the invention, mention can particularly be made of the following compounds:
C-beta-D-xylopyranoside-n-propane-2-one;
C-alpha-D-xylopyranoside-n-propane-2-one;
C-beta-D-xylopyranoside-2-hydroxy-propane;
C-alpha-D-xylopyranoside-2-hydroxy-propane-;
1-(C-beta-D-glucopyranosyl)-2-hydroxy-propane;
1-(C-alpha-D-glucopyranosyl)-2-hydroxy-propane;
1-(C-beta-D-glucopyranosyl)-2-amino-propane;
1-(C-alpha-D-glucopyranosyl)-2-amino-propane;
3′-(acetamido-C-beta-D-glucopyranosyl)-propane-2′-one;
3′-(acetamido-C-alpha-D-glucopyranosyl)-propane-2′-one;
1-(acetamido-C-beta-D-glucopyranosyl)-2-hydroxyl-propane;
1-(acetamido-C-beta-D-glucopyranosyl)-2-amino-propane;
as well the cosmetically acceptable salts thereof, solvates thereof such as hydrates and the optical isomers thereof.

Preferably, C-beta-D-xylopyranoside-2-hydroxy-propane or C-alpha-D-xylopyranoside-2-hydroxy-propane is used, and more preferably C-beta-D-xylopyranoside-2-hydroxy-propane. Preferably, a C-glycoside of formula (I) suitable for the invention can be advantageously C-beta-D-xylopyranoside-2-hydroxypropane, of which the lNCl name is HYDROXYPROPYL TETRAHYDROPYRANTRIOL particularly sold under the trade name MEXORYL SBB® or MEXORYL SCN® by CHIMEX. The salts of the C-glycosides of formula (I) suitable for the invention can comprise conventional physiologically acceptable salts of these compounds such as those formed from organic or inorganic acids. By way of example, mention can be made of the salts of mineral acids, such as sulfuric acid, hydrochloric acid, hydrobromic acids, hydroiodic acid, phosphoric acid and boric acid. Mention can also be made of organic salts, which can include one or more carboxylic, sulfonic, or phosphonic acid groups. They can consist of linear, branched or cyclic aliphatic acids or indeed aromatic acids. These acids can further include one or more heteroatoms selected from O and N, for example in the form of hydroxyl groups. Mention can particularly be made of propionic acid, acetic acid, terephthalic acid, citric acid, and tartaric acid.

The acceptable solvates for the compounds described above comprise conventional solvates such as those formed during the final preparation step of said compounds due to the presence of solvents. By way of example, mention can be made of solvates due to the presence of water or linear or branched alcohols such as ethanol or isopropanol.

The C-glycosides (I) are known from the document WO 02/-1051828.

According to an embodiment, the composition according to the invention comprises a C-glycoside in a quantity between 0.05% and 10% by weight of active substance (C-glycoside) with respect to the total weight of the composition, in particular between 0.5% and 7.5% by weight of active substance with respect to the total weight of the composition, more specifically between 1% and 5% by weight of active substance with respect to the total weight of the composition.

The compositions according to the invention can contain one or more of the usual adjuvants in the cosmetic and dermatological fields, gelling agents and/or lipophilic thickeners; moisturizing agents; emollients; lipophilic active agents; anti-free radical agents; sequestrants; fillers; antioxidants, such as pentaerythrityl tetra-di-t-butyl hydroxyhydrocinnamate; essential oils; perfumes; film-forming agents; soluble dyes; and mixtures thereof. The quantities of these different adjuvants are those conventionally used in the fields in question. In particular, these quantities vary according to the sought purpose and can for example range from 0.01% to 20%, and preferably from 0.1% to 10% by weight of the total weight of the composition.

Obviously, those skilled in the art will take care to choose the optional adjuvant(s) added to the composition according to the invention in such a way that the advantageous properties intrinsically associated with the composition according to the invention are not altered, or are not substantially altered, by the envisaged addition.

By way of gelling agents and/or lipophilic thickeners, mention can be made of esters of dextrin and fatty acids, in particular dextrin palmitates such as for example those sold under the trade name RHEOPEARL TL2-OR or RHEOPEARL KL2-OR by CHIBA FLOUR MILLING, and under the trade name RHEOPEARL KS by CHIBA FLOUR MILLING, and dextrin myristates, such as for example those sold under the trade name RHEOPEARL MKL2 by CHIBA FLOUR MILLING. Mention can further be made of tri-esters of fatty acids and mono or polyglycerol such as glyceryl tri-(hydroxystearate) (INCI name: TRIHYDROXYSTEARIN) such as for example that sold by ELEMENTIS under the trade name THIXClN R or that which is sold by BYK ADDITIVES & INSTRUMENTS under the trade name RHEOCIN; modified clays such as hectorite and derivatives thereof, such as the products under the names Bentone.

Preferentially, the composition comprises less than 3% by weight of gelling agent and/or lipophilic thickener, preferably less than 2% by weight of gelling agent and/or lipophilic thickener with respect to the total weight of the composition, more preferably, less than 1% by weight of gelling agent and/or lipophilic thickener with respect to the total weight of the composition. In a specific embodiment, the composition is free from gelling agent and/or lipophilic thickener.

The composition according to the invention can further comprise additional active agents different from the hydrophilic active agents cited above.

The quantities of these different active agents are those conventionally used in the fields in question. In particular, these quantities vary according to the sought purpose and can for example range from 0.01% to 20%, and preferably from 0.1% to 10% by weight of the total weight of the composition.

Obviously, those skilled in the art will take care to choose the optional active agent(s) added to the composition according to the invention in such a way that the advantageous properties intrinsically associated with the composition according to the invention are not altered, or are not substantially altered, by the envisaged addition.

The composition can further comprise one or more UV filters, in particular organic filters, particularly liposoluble organic filters.

According to a particular embodiment, the composition according to the invention comprises less than 2% by weight of filler with respect to the total weight of the composition, more preferably, the composition according to the invention is free from filler, The term “filler” should be understood to denote inorganic or synthetic, colorless or white particles of any shape, insoluble in the medium of the composition regardless of the temperature at which the composition is manufactured.

The composition according to the invention also relates to the cosmetic use of a composition according to the invention for the care of keratin materials, in particular for body and/or facial skin care.

The present invention further relates to a non-therapeutic cosmetic care method of keratin materials such as the skin, in particular body and/or facial skin, wherein a composition according to the invention is applied on said keratin materials.

The composition according to the invention can be obtained conventionally by those skilled in the art.

The following examples make it possible to understand the invention better, without being in any way limitative. The raw materials are referred to with the chemical or INCI name thereof. The quantities are indicated by weight of raw materials with respect to the total weight of the composition, unless specified otherwise.

### Example 1: Preparations of Compositions According To The Invention And of Comparative Compositions, and Evaluation of the Macroscopic Appearance Thereof

Compositions F1 to F10, explained in tables 1 to 3, were prepared according to the following method:
a) Produce a pre-dispersion of the hydrophilic active agent in the mixture of glycols and monoalcohol under magnetic stirring or a deflocculator until a perfectly homogeneous and clear mixture is obtained; then
b) Add the oily phase until a perfectly homogeneous and clear mixture is obtained.

Compositions F1 to F6, F8 and F9 are according to the invention, whereas compositions F7 and F10 are comparative (marked by two asterisks).

Table 1

| | F1 | F2 | F3 | F4 |
|---|---|---|---|---|
| Octyldodecanol (Eutanol G®, BASF) | qs 100 | qs 100 | qs 100 | qs 100 |
| Castor oil (Crystal O®, Vertellus) | 3.7 | 9 | 13.19 | 17.38 |
| Oleic alcohol (HD OCENOL 80/85 V/MB®, BASF) | 4.94 | 4.81 | 4.81 | 4.81 |
| Camelina oil (CAMELINA REFINED OIL C05002®, GREENTECH) | 1.98 | 1.92 | 1.92 | 1.92 |
| Pentaerythrityl tetra-di-t-butyl hydroxyhydrocinnamate (Tinogard TT, BASF) | | 0.1 | 0.1 | 0.1 |
| Hydroxypropyl tetrahydropyrantriol (MEXORYL SCN®, CHIMEX) (35% AS* in water)) | 8.7 (= 3.045% AS) | 8.7 (= 3.045% AS) | 8.7 (= 3.045% AS) | 8.7 (= 3.045% AS) |
| Hexylene qlycol | 10 | 9 | 8 | 7 |
| Dipropylene glycol | 16.9 | 15.21 | 13.52 | 11.83 |
| Ethanol | 15 | 13.5 | 12 | 10.5 |

* AS: active substance

Table 2

| | F5 | F6 | F7** |
|---|---|---|---|
| Octyldodecanol (Eutanol G®, BASF) | qs 100 | qs 100 | qs 100 |
| Castor oil (Crystal O®, Vertellus) | 21.57 | 25.58 | 36.05 |
| Oleic alcohol (HD OCENOL 80/85 V/MB®, BASF) | 4.81 | 4.94 | 4.94 |
| Camelina oil (CAMELINA REFINED OIL C05002®, GREENTECH) | 1.92 | 1.97 | 1.97 |
| Pentaerythrityl tetra-di-t-butyl | 0.1 | 0.1 | 0.1 |
| hydroxyhydrocinnamate (Tinogard TT, BASF) | | | |
| Hydroxypropyl tetrahydropyrantriol (MEXORYL SCN®, CHIMEX) (35% AS* in water)) | 8.7 (= 3.045% AS) | 8.7 (= 3.045% AS) | 8.7 (= 3.045% AS) |
| Hexylene glycol | 6 | 5 | 2.5 |
| Dipropylene glycol | 10.14 | 8.45 | 4.23 |
| Ethanol | 9 | 7.5 | 3.75 |

* AS: active substance

Table 3

| | F8 | F9 | F10** |
|---|---|---|---|
| Octyldodecanol (Eutanol G®, BASF) | qs 100 | qs 100 | qs 100 |
| Castor oil (Crystal O®, Vertellus) | 3.61 | 25.11 | 35.9 |
| Oleic alcohol (HD OCENOL 80/85 V/MB®, BASF) | 4.81 | 4.81 | 4.81 |
| Camelina oil ( CAMELlNA REFINED OIL C05002®, GREENTECH) | 1.92 | 1.92 | 1.92 |
| Pentaerythrityl tetra-di-t-butyl hydroxyhydrocinnamate (Tinogard TT, BASF) | | 0.1 | 0.1 |
| Hydroxypropyl tetrahydropyrantriol (MEXORYL SCN®, CHIMEX) (35% AS* in water)) | 8.7 (= 3.045% AS) | 8.7 (= 3.045% AS) | 8.7 (= 3.045% AS) |
| Hexylene glycol | 8.7 | 4.35 | 2.18 |
| Dipropylene glycol Ethanol | 21.4 | 10.7 | 5.35 |
| | 13.1 | 6.55 | 3.28 |

* AS: active substance

The macroscopic appearance for each composition was evaluated at ambient temperature 24 hours after manufacture. The homogeneity (single-phase or bi-phase) and the clearness (transparent or turbid) are also evaluated, and the results are in tables 4 and 5.

Tables 4 and 5 also include the weight ratio of oily phase to hydrophilic phase.

The oily phase includes all the oils, whereas the hydrophilic phase includes the aliphatic monoalcohol, dipropylene glycol, hexylene glycol, water and the hydrophilic active agent.

Table 4

| | F1 | F2 | F3 | F4 | F5 | F6 | F7** |
|---|---|---|---|---|---|---|---|
| Macroscopic appearance | Transparent single-phase | | | | | Borderline transparent single-phase | Turbid bi-phase |
| Weight ratio of oily phase to hydrophilic phase | 0.98 | 1.15 | 1.37 | 1.63 | 1.96 | 2.37 | 4.22 |

Table 5

| | F8 | F9 | F10** |
|---|---|---|---|
| Macroscopic appearance | Transparent single-phase | Borderline transparent single-phase | Turbid bi-phase |
| Weight ratio of oily phase to hydrophilic phase | 0.93 | 2.30 | 4.13 |

Conclusions: therefore, the results show that, to obtain a composition according to the invention, i.e., single-phase and transparent, the quantity of ethanol must be between 6% and 15% by weight with respect to the total weight of the composition; the quantity of hexylene glycol must be between 4% and 10% by weight with respect to the total weight of the composition; and the quantity of dipropylene glycol must be between 8% and 25% by weight with respect to the total weight of the composition.

### Example 2: Evaluation of Compositions F1 and F8 and of a Comparative Composition F. on Skin Finish using Skin Fx® Type Reconstructed Skins

The sensory properties of compositions F1 and F8 from example 1 (reproduced in table 6), and of a comparative composition F described in table 6, were evaluated by a panel of 3 subjects.

Table 6

| | F comparative | F1 | F8 |
|---|---|---|---|
| Octyldodecanol (Eutanol G®, BASF) | qs 100 | qs 100 | qs 100 |
| Castor oil (Crystal O®, Vertellus) | 7.5 | 3.7 | 3.61 |
| Oleic alcohol (HD OCENOL 80/85 V/MB®, BASF) | 10 | 4.94 | 4.81 |
| Camelina oil (CAMELINA REFINED OIL C05002®, GREENTECH) | 4 | 1.98 | 1.92 |
| Hydroxypropyl tetrahydropyrantriol (MEXORYL SCN®, CHIMEX) (35% AS*)) | 8.67 (= 3.035% AS) | 8.7 (= 3.045% AS) | 8.7 (= 3.045% AS) |
| Glycerin | 1.5 | | |
| Pentylene glycol | 23.35 | | |
| Hexylene glycol | | 10 | 8.7 |
| Dipropylene glycol | | 16.9 | 21.4 |
| Ethanol | 15 | 15 | 13.1 |

* AS: active substance

For each composition, a layer of 35 µL was applied on a Skin FX neutral substrate (silicone-based substrate having a diameter of 80 mm and a surface area of 35 cm², coated with a polyurethane film, imitating skin texture). This layer, particularly in terms of thickness, is similar to a layer resulting from the conventional application of a composition by a user.

The application is carried out as follows:
Deposit the product on the substrate
Spread the product around with the fingers 15 times in 15 seconds (evaluation of slip)
Wait 15 seconds
Continue the application by spreading around 15 more times in 15 seconds (evaluation of butter grease feel on fingers)
Wait 2 minutes
Evaluation of the criterion below.

The criterion of the greasy feel on the fingers during application was evaluated; it helps qualify the emollience of the formulas.

Specifically, the criterion of the greasy feel on the fingers during application was evaluated on a scale of 1 to 5 (1 corresponding to “sightly” and 5 corresponding “considerably/very”):

For the comparative composition, the criterion of the greasy feel on the fingers during application is 1.68.

For composition F1, the criterion of the greasy feel on the fingers during application is 2.67. For the composition F8, the criterion of the greasy feel on the fingers during application is 2.0.

The values indicated above correspond to a mean calculated on 3 subjects. The acceptable standard deviation being less than or equal to 1.0.

The above results show that replacing pentylene glycol by the mixture of hexylene glycol and dipropylene glycol according to the invention increases the butter grease feel skin finish of the compositions, and therefore enhances emollience.

### Example 3: Effects of Compositions F1 And F8 and of a Comparative Composition F, on the Penetration of the Hydrophilic Active Agent

Compositions F1 and F8 from example 1 were compared to comparative composition F from example 2 (table 6), in respect of the penetration of the hydrophilic active agent hydroxypropyl tetrahydropyrantriol (MEXORYL SCN® from CHIMEX) in the skin. Comparative composition F enables a very good penetration of the hydrophilic active agent.

The protocol is as follows:

Compositions F1, F8 or the reference composition, are applied on pig skin samples at a rate of 20 mg/cm² for 10 minutes.

After a 24-hour period following the application of the composition, the quantity of active agent present in the recipient fluid or in the skin (epidermis and dermis) is analyzed.

The results are as follows:

Table 7

| Hydrophilic active agent in (Epidermis+Dermis+Recipient Fluid) | Comparative composition F | F8 | F1 |
|---|---|---|---|
| µg/cm² | 29501 ±15100 | 26021 ±9541 | 25045 ±11362 |
| % Dose applied | 20.08 ±10.9 | 17.67 ±6.61 | 17.22 ±7.83 |

They show that compositions F1 and F8 enable similar penetration of the hydrophilic active agent in the skin to the comparative composition.

### Example 4: Composition F11 According to the Invention

Composition F11 according to the invention is prepared as described in example 1.

Table 8

| | F11 |
|---|---|
| Octyldodecanol (Eutanol G®, BASF) | qs 100 |
| Castor oil (Crystal O®, Vertellus) | 3.6 |
| Oleic alcohol (HD OCENOL 80/85 V/MB®, BASF) | 4.8 |
| Camelina oil (CAMELINA REFINED OIL C05002®, GREENTECH) | 1.9 |
| Hydroxypropyl tetrahydropyrantriol (MEXORYL SCN®, CHIMEX) (35% AS* in water) | 8.7 (= 3.045% AS) |
| Hexylene glycol | 4.4 |
| Dipropylene glycol | 10.7 |
| Ethanol | 6.6 |

*AS: active substance

### Example 5: Comparative Composition

The following comparative composition (i.e. without dipropylene glycol) is prepared as described in example 1:

Table 9

| | Comparative formula |
|---|---|
| Hydroxypropyl tetrahydropyrantriol (MEXORYL SCN®, CHIMEX) (35% AS* in water) | 2.86 (= 1% AS) |
| Oleic alcohol | 10 |
| Isopropyl palmitate | Qsp 100 |
| Hexylene glycol | 10 |
| Dipropylene glycol | |

* AS: active substance

After preparation, the comparative composition turns trouble, and results in a biphase composition after 24 h.

Thus, in the absence of dipropylene glycol, stability is altered.

1. A cosmetic composition comprising:
from 30% to 89% by weight of at least one polar oil with respect to the total weight of the composition;
from 6% to 45% by weight of at least one C₂-C₆ aliphatic monoalcohol with respect to the total weight of the composition;
from 4% to 30% by weight of hexylene glycol with respect to the total weight of the composition;
from 8% to 40% by weight of dipropylene glycol with respect to the total weight of the composition; and at least one hydrophilic active agent,
said composition comprising less than 15% by weight of water with respect to the total weight of the composition.

2. The composition according to claim 1, which comprises from 35% to 80% by weight of at least one polar oil- with respect to the total weight of the composition.

3. The composition according to claim 1, wherein the polar oil is a non-volatile polar oil.

4. The composition according to claim 1, wherein the polar oil(s) is/are selected from triglycerides constituted of esters of glycerol and C₄ to C₂₄ fatty acids that are linear or branched, saturated or unsaturated; esters of isopropyl alcohol and C₈-C₁₈ fatty acids; fatty alcohols that are liquid at ambient temperature, with a branched and/or unsaturated carbon chain having from 16 to 22 carbon atoms ; dialkyl carbonates, both alkyl chains being identical; diesters of C₈-C₁₂ dicarboxylic acid and branched C₃-C₄ monoalcohol; and mixtures thereof.

5. The composition according to claim 1, which comprises at least two polar oils, different from one another, of which at least a first polar oil is selected from fatty alcohols that are liquid at ambient temperature, with a branched and/or unsaturated carbon chain having from 18 to 20 carbon atoms, and at least a second polar oil is selected from triglycerides of glycerol and C₁₄-C₂₂ fatty acids.

6. The composition according to claim 1, which comprises a mixture of octyldodecanol, oleic alcohol, castor oil and camelina oil.

7. The composition according to claim 5, wherein the first polar oil selected from fatty alcohols that are liquid at ambient temperature, with a branched and/or unsaturated carbon chain having from 18 to 20 carbon atoms, is present in the composition at a concentration between 25% and 65% by weight with respect to the total weight of the composition; and/or the second polar oil selected from triglycerides of glycerol and C₁₄-C₂₂ fatty acids is present in the composition at a concentration between 4% and 30% by weight with respect to the total weight of the composition.

8. The composition according to claim 1, wherein the C₂-C₆ aliphatic monoalcohol is selected from the group consisting of ethanol, propanol, butanol, isopropanol, isobutanol and mixtures thereof.

9. The composition according to claim 1, which comprises from 4% to 10% by weight of hexylene glycol, and/or from 8% to 25% by weight of dipropylene glycol with respect to the total weight of the composition.

10. The composition according to claim 1, wherein the hydrophilic active agent is selected from C-glycoside derivatives of the following general formula (I): 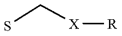
wherein:
R designates a non-substituted C₁-C₄ linear alkyl radical ;
S represents a monosaccharide selected from the group consisting of D-glucose, D-xylose, N-acetyl-D-glucosamine and L-fucose;
X represents a group selected from the group consisting of —CO—, —CH(OH)— and —CH(NH₂)—; the cosmetically acceptable salts thereof, solvates thereof and the optical isomers thereof.

11. Composition according to claim 1, wherein the hydrophilic active agent is selected from the group consisting of C-beta-D-xylopyranoside-2-hydroxy-propane and C-alpha-D-xylopyranoside-2-hydroxy-propane.

12. The composition according to claim 1, which comprises less than 8% by weight of water with respect to the total weight of the composition.

13. (canceled)

14. A non-therapeutic cosmetic care method for keratin materials, comprising the application on said keratin materials of a composition according to claim 1.

15. The composition according to claim 2 wherein the polar oil is a non-volatile polar oil.

16. The composition according to claim 2 wherein the polar oil(s) is/are selected from triglycerides constituted of esters of glycerol and C₄ to C₂₄ fatty acids that are linear or branched, saturated or unsaturated; esters of isopropyl alcohol and C₈-C₁₈ fatty acids; fatty alcohols that are liquid at ambient temperature, with a branched and/or unsaturated carbon chain having from 16 to 22 carbon atoms; dialkyl carbonates, both alkyl chains being identical; diesters of C₈-C₁₂ dicarboxylic acid and branched C₃-C₄ monoalcohol; and mixtures thereof.

17. The composition according to claim 3 wherein the polar oil(s) is/are selected from triglycerides constituted of esters of glycerol and C₄ to C₂₄ fatty acids that are linear or branched, saturated or unsaturated; esters of isopropyl alcohol and C₈-C₁₈ fatty acids; fatty alcohols that are liquid at ambient temperature, with a branched and/or unsaturated carbon chain having from 16 to 22 carbon atoms; dialkyl carbonates, both alkyl chains being identical; diesters of C₈-C₁₂ dicarboxylic acid and branched C₃-C₄ monoalcohol; and mixtures thereof.

18. The composition according to claim 2, which comprises at least two polar oils, different from one another, of which at least a first polar oil is selected from fatty alcohols that are liquid at ambient temperature, with a branched and/or unsaturated carbon chain having from 18 to 20 carbon atoms, and at least a second polar oil is selected from triglycerides of glycerol and C₁₄-C₂₂ fatty acids.

19. The composition according to claim 3, which comprises at least two polar oils, different from one another, of which at least a first polar oil is selected from fatty alcohols that are liquid at ambient temperature, with a branched and/or unsaturated carbon chain having from 18 to 20 carbon atoms, and at least a second polar oil is selected from triglycerides of glycerol and C₁₄-C₂₂ fatty acids.

20. The composition according to claim 2, which comprises at least two polar oils, different from one another, of which at least a first polar oil is selected from fatty alcohols that are liquid at ambient temperature, with a branched and/or unsaturated carbon chain having from 18 to 20 carbon atoms, and at least a second polar oil is selected from triglycerides of glycerol and C₁₄-C₂₂ fatty acids.

21. The composition according to claim 2 which comprises a mixture of octyldodecanol, oleic alcohol, castor oil and camelina oil.
